(19) United States
(12) Patent Application Publication
(10) Pub. No.: US 2013/0224164 A1
(43) Pub. Date: Aug. 29, 2013

(54) Environmental Clostridial Bacteriotherapy and Related Formulations and Methods of Manufacture and Use

(71) Applicants: Walter Tatarowicz, Exton, PA (US); Colin Broom, Devon, PA (US)

(72) Inventors: Walter Tatarowicz, Exton, PA (US); Colin Broom, Devon, PA (US)

(73) Assignee: VIROPHARMA INCORPORATED, Exton, PA (US)

(21) Appl. No.: 13/821,801

(22) PCT Filed: Sep. 7, 2011
(86) PCT No.: PCT/US11/50657

§ 371 Date: May 20, 2013

Related U.S. Application Data
(60) Provisional application No. 61/381,693, filed on Sep. 10, 2010.

(51) Int. Cl. A61K 35/74; A61K 31/7048; A61K 38/14

(52) U.S. Cl. CPC A61K 35/74; A61K 38/14; A61K 31/7048
USPC 424/93.41; 514/3.1; 514/28

(57) ABSTRACT

Compositions and methods for inhibiting Clostridium associated diseases are disclosed.

This application claims priority under 35 U.S.C. §119(e) to U.S. Provisional Patent Application No. 61/381,693, filed Sep. 10, 2010. The foregoing application is incorporated by reference herein.

### FIELD OF THE INVENTION

The present invention relates to the field of bacteriotherapy. More specifically, the invention provides compositions and methods for the inhibition of Clostridium disease.

### BACKGROUND OF THE INVENTION

Several publications and patent documents are cited throughout the specification in order to describe the state of the art to which this invention pertains. Each of these citations is incorporated herein by reference as though set forth in full.

Clostridium infections are a major burden on health care facilities, producing both endemic and epidemic diarrhea with significant morbidity and mortality (Bauer et al. (2009) Curr. Opin. Infect. Dis., 22:517-524; Dallal et al. (2002) Ann. Surg., 235:363-372; Pepin et al. (2004) CMAJ, 171:466-472; Loo et al. (2005) N. Engl. J. Med., 353:2442-2449; Labbe et al. (2008) Antimicrob. Agents Chemother., 52:3180-3187; Kuijper et al. (2007) Euro. Surveill., 12:E1-E2; Kuijper et al. (2006) Clin. Microbiol. Infect., 12:2-18). Aerosolization of Clostridium has recently been demonstrated (Best et al. (2010) Clin. Infect. Dis., 50:1450-1457; Roberts et al. (2008) BMC Infect. Dis., 8:7). Indeed, Best et al. demonstrate that Clostridium difficile is commonly but sporadically present in the air around symptomatic patients with C. difficile infection. Considering the impracticability of isolating all patients with a Clostridium infection, there is a clear need for improved methods of inhibiting Clostridium associated diseases.

### SUMMARY OF THE INVENTION

According to one aspect of the instant invention, methods of inhibiting disease caused by Clostridium in a subject are provided. In a particular embodiment, the method comprises providing a first subject that has been administered a non-toxigenic strain of Clostridium and is shedding the non-toxigenic Clostridium; administering at least one antibiotic to a second subject; and exposing the second subject to the first subject, whereby the exposure of the second subject to the first subject results in the colonization of the gastrointestinal tract of the second subject by the non-toxigenic Clostridium. In a particular embodiment, the Clostridium is C. difficile or C. butyricum. The first and second subject may individually be a human or animal. The first and second subject may occupy the same environment (e.g., room) at the same time or consecutively (wherein the first subject is in the environment first). The first and second subjects may or may not have direct physical contact.

According to another aspect of the instant invention, methods of inhibiting disease caused by Clostridium in a subject are provided comprising administering at least one antibiotic to the subject and contacting the environment of the subject with an effective amount of a non-toxigenic strain of Clostridium. In a particular embodiment, the subject is maintained in the environment for a time sufficient to allow the colonization of the gastrointestinal tract of the subject by the non-toxigenic Clostridium. In a particular embodiment, the Clostridium is C. difficile or C. butyricum.

According to another aspect of the instant invention, methods of producing a non-toxigenic strain of Clostridium are provided. In a particular embodiment, the method comprises administering to an animal host a sufficient quantity of the non-toxigenic strain of Clostridium to induce colonization of the gastrointestinal tract of the host, thereby causing shedding of the spores by the host. The method may further comprise exposing the host to a subject in order to effect transfer of the non-toxigenic Clostridium from the host to the subject. The exposure to the shed spores results in the inhibition of disease caused by Clostridium in the subject. In a particular embodiment, the transfer of the non-toxigenic Clostridium occurs by exposing the subject to the same environment as the host. In yet another embodiment, the transfer of the non-toxigenic Clostridium occurs by applying the shed spores from the host (optionally isolated) to the environment of the subject.

According to still another aspect of the instant invention, methods of protecting a patient undergoing medical treatment at a treatment site from acquiring a disease caused by a Clostridial infection while present at the site are provided. In a particular embodiment, the method comprises dispersing a non-toxic strain of Clostridium at the site in an amount sufficient to be transferred to the patient, thereby effecting colonization of the gastrointestinal tract of the patient by the non-toxigenic Clostridium.

According to yet another aspect of the instant invention, methods are provided for reducing the risk that a medical treatment site will induce a disease caused by a Clostridial infection in a patient upon undergoing treatment at the site. In a particular embodiment, the method comprises dispersing a non-toxic strain of Clostridium at the site in an amount that is sufficient to be transferred to a patient exposed to the site, to thereby effect colonization of the gastrointestinal tract of the patient by the non-toxigenic strain of Clostridium.

### BRIEF DESCRIPTION OF THE DRAWINGS

FIGS. 1A-1C provide the C. difficile stool culture results for cohort 1 (placebo or 10⁴ spores), cohort 2 (placebo or 10⁶ spores), and cohort 3 (placebo or 10⁸ spores), respectively. Baseline=study day prior to start of dosing with oral vancomycin (Study Days −5 to −1). ND=not done (stool sample not available). Stool culture results for C. difficile: + (positive) or − (negative). (p)=Toxin A/B positive. (n)=Toxin A/B negative. Shaded=C. difficile genotype consistent with VP 20621.

### DETAILED DESCRIPTION OF THE INVENTION

The instant invention relates to the discovery that Clostridial bacteriotherapy may be administered to a host in need of treatment via secondary/environmental dosing. According to one aspect of the invention, the environmental dosing is from a host (vector system) that has been previously administered a desired Clostridial spore containing formulation. Therefore, the host may be the in vivo manufacturer/producer of the desired bacteriotherapy formulation. According to another embodiment, the method of patient/environmental dosing is achieved by application of a Clostridium formulation (e.g., spores) that is manufactured by in vitro culture methods to the environment inhabited by the patient.

According to the instant invention, the methods of preventing/inhibiting a toxigenic Clostridium (e.g., C. difficile) infection and the diseases/disorders associated therewith, in a patient are provided. In accordance with the instant invention, at least one non-toxigenic Clostridium is administered to a host. In a particular embodiment, the non-toxigenic Clostridium is administered at a concentration appropriate to colonize the gastrointestinal tract of the host and cause shedding of the non-toxigenic Clostridium. In a particular embodiment, the method comprises first dosing a patient with an antibiotic (e.g., oral vancomycin (VANCOCIN®)), and then dosing the patient with a non-toxigenic Clostridium (e.g., spores of the M3 strain of C. difficile). The non-toxigenic Clostridium may be obtained and transferred to the patient from a host (e.g. a human or animal vector system) that has been dosed directly (or indirectly) with the non-toxigenic Clostridium (e.g., spores of the M3 strain of C. difficile). The non-toxigenic Clostridium may be transferred directly to the patient (e.g., by physical contact and/or sharing of bodily fluids) from the host. The non-toxigenic Clostridium may also be transferred indirectly to the patient from the environment inhabited by the patient and the host. Transfer to the patient may be accomplished via host contact with surfaces, substances or fluids that also come in contact with the patient, or via host induced aerosolized non-toxigenic Clostridium material into the environment shared with the patient. The patient and the host may inhabit the same environment at the same or at different times.

The instant invention encompasses bacteriotherapy that uses non-toxigenic or substantially non-toxigenic Clostridium. In a particular embodiment, the Clostridium is C. difficile or C. butyricum. The non-toxigenic strain of Clostridium may be, for example, a C. difficile strain selected from one or more of the M, T, C, P, S and AP groups in accordance with the REA typing system for C. difficile. In a particular embodiment, the C. difficile strain is selected from the group consisting of M, M3, M23, T, T1, T7, C, P, S, and AP. In still another embodiment, the non-toxigenic C. difficile is from the M group, particularly M3 or M23, or T group, particularly T7. Restriction endonuclease analysis (REA) may be used to type isolates (see, e.g., Clabots et al. (1993) J. Clin. Microbiol., 31: 1870-1875 and U.S. Pat. No. 6,635,260). In a particular embodiment, the Clostridial species is a non-toxigenic M3 strain of C. difficile (e.g., VP20621) or C. butyricum MIYAIRI 588 (CBM 588).

Without wishing to be bound by any particular theory, the ability of a non-toxigenic strain to protect against Clostridium associated disease is believed to correlate with the ability of the non-toxigenic strain to colonize the gut/gastrointestinal tract. Accordingly, the most frequently isolated REA types from humans may be the best at colonizing the human gut. However, non-toxigenic strains identified in one animal (e.g., human) may effectively colonize different species (e.g., nonhumans).

In accordance with the instant invention, methods of using a host-vector/manufacturing system are provided. The invention encompasses methods of manufacturing Clostridial bacteriotherapy formulations that are then used to dose patients or dose the environment that will be inhabited by the patient. In a particular embodiment, the methods comprise manufacturing a non-toxigenic Clostridium formulation by isolating Clostridium spores from a host (e.g., human) and delivering the non-toxigenic Clostridium formulation (e.g., at least one non-toxigenic Clostridium spore/cell and at least one carrier) to a patient's environment.

The instant invention encompasses methods of delivering the non-toxigenic Clostridium material to the patient's environment. The methods include but are not limited to placing non-toxigenic Clostridium material on surfaces and/or into substances or fluids (other than a traditional oral medicinal preparation) that may come in contact with the patient to be treated. The methods also may include aerosolizing non-toxigenic Clostridium material in the patient's environment or delivering aerosolized non-toxigenic Clostridium material into the patient's environment. The patient contact with aerosolized material may be from the host, or may be independently from aerosolization of Clostridium material using the appropriate aerosolization device.

A further aspect of the invention is the use of specific measures to focus and limit dissemination of the bacteriotherapy to the desired patient population. The bacteriotherapy preferably may be focused to target patient populations (or treatment facilities and locations) by using contact precautions (e.g. limiting contact with surfaces, substances and fluids that may contain the non-toxigenic Clostridium material). Ventilation and air filtration devices may be designed and configured to focus and limit exposure to the indicated bacteriotherapy to the desired target patient population or treatment location. The methods of the instant invention include use of the bacteriotherapy only in the locations where there is a desired patient population to be treated. The methods of the instant invention include the distribution and use of labeling, packaging and instructional materials that contain information to guide the proper and desired use of the bacteriotherapy and to promote or achieve the invention objectives.

The carrier used with the non-toxigenic Clostridium spore may be pharmaceutically acceptable or pharmaceutically unacceptable. For example, for application of the non-toxigenic Clostridium spore directly to the environment of the patient, the carrier may be any carrier that is not incompatible with the non-toxigenic Clostridium spore (i.e., the carrier does not prevent the non-toxigenic Clostridium spores from being viable). In a particular embodiment, the non-toxigenic Clostridium spores are contained within a carrier which comprises preservatives, antimicrobials, and the like which are not suitable for administration to a human or animal. Except insofar as any conventional media or agent is incompatible with the non-toxigenic Clostridium spores, its use as a carrier is contemplated. In a particular embodiment, the carrier promotes the dispersion of the non-toxigenic Clostridium spores into the environment in which the non-toxigenic Clostridium formulation is applied.

In another embodiment, the methods comprise administering to a human host a sufficient quantity of non-toxigenic Clostridium formulation to induce colonization of the host and then placing the host in the patient's environment, particularly during the time of Clostridium shedding by the host. In a particular embodiment, the host may be administered at least one antibiotic prior to administration of the non-toxigenic Clostridium in order to create a more receptive environment for colonization with the non-toxigenic Clostridium. The patient may also be administered at least one antibiotic prior to exposure to the environmental exposure to the non-toxigenic Clostridium in order to create a more receptive environment for colonization with the non-toxigenic Clostridium. The host used to manufacture the non-toxigenic Clostridium may be a human or animal. The host may be healthy or may be a patient/subject under treatment for infection or some other health problem. The host may inhabit the same environment before or concomitantly with the patient. The host may be, without limitation, a healthcare provider, another patient, family member, friend or pet.

In a particular embodiment, the subject is exposed to the environmental dosing (e.g., exposed to a host shedding Clostridium, exposed to an environment previously occupied by a host shedding Clostridium, and/or exposed to an environment containing applied Clostridium) for at least 12 hours, for at least 1, 2, 3, or more days, or for at least 1, 2, 3, 4 or more weeks. In a particular embodiment, the colonization by the non-toxigenic Clostridium occurs within about 12, 24, 48, 72, 96, or more hours of exposure.

The instant invention encompasses methods of reducing the risk that a medical (or non-medical) treatment site will induce a disease caused by a Clostridium infection wherein the method includes the step of administering a therapeutically effective amount of a non-toxigenic Clostridium bactereotherapy to a patient at risk of contracting said disease. A preferred embodiment of the invention is wherein the risk is reduced by more than 50%, more than 75% or more than 90% in the medical treatment location. A preferred feature of the invention is wherein the risk is reduced within about 3, 2, 1 or less weeks (and more preferably within about 24, 12, 6, 3, 1 or less hours) of initiating the bacteriotherapy at the medical treatment site.

The methods described herein may be used alone or in conjunction to generally prevent/inhibit Clostridial infections in a healthcare facility (e.g., hospital). For example, workers at the health care facility may be directly treated with the non-toxigenic Clostridium and/or the physical environment of the healthcare facility may be dosed with non-toxigenic Clostridium. As such, the health care facility becomes safe for patients who are at risk from toxigenic/life threatening Clostridial infections by treatment of the hospital environment with the desired beneficial bacteriotherapy.

As explained hereinabove, the host may be administered at least one antibiotic prior to administration of the non-toxigenic Clostridium. In another embodiment, the patient is administered at least one antibiotic prior to environmental exposure to the non-toxigenic Clostridium. In a particular embodiment, the antibiotic(s) is administered orally. The non-toxigenic Clostridium may be administered at any time after the antibiotic treatment. The subject may be delivered/exposed to the non-toxigenic Clostridium within 96 hours, particularly within 72, 48, or 24 hours, of the administration of the antibiotic. The subject may be delivered/exposed to the non-toxigenic Clostridium at least one hour, particularly at least 4, 8, or 12 hours after the administration of the antibiotic. In a particular embodiment, the host is delivered at least 1 spore, at least 10 spores, at least 10² spores, at least 10³ spores, at least 10⁴ spores, at least 10⁵ spores, at least 10⁶ spores, at least 10⁷ spores, at least 10⁸ spores, at least 10⁹ spores or more in one or more doses. The doses may be administered more than once a day and over a course of days (e.g., over 3, 5, 7, 10, 14, or more days). The non-toxigenic Clostridium may be administered at appropriate intervals and doses to first establish a colonization of the gastrointestinal tract, after which the dosage may be reduced to a maintenance level to maintain the colonization and shedding of spores.

Antibiotics of the instant invention include, without limitation, beta-lactams (e.g., penicillin, ampicillin, oxacillin, cloxacillin, methicillin, and cephalosporin), carbacephems, cephamycins, carbapenems, monobactams, aminoglycosides (e.g., gentamycin, tobramycin), glycopeptides (e.g., vancomycin), quinolones (e.g., ciprofloxacin), moenomycin, tetracyclines, macrolides (e.g., erythromycin), fluoroquinolones, oxazolidinones (e.g., linezolid), lipopetides (e.g., daptomycin), aminocoumarin (e.g., novobiocin), co-trimoxazole (e.g., trimethoprim and sulfamethoxazole), lincosamides (e.g., clindamycin and lincomycin), nitroimidazole (e.g., metronidazole), polypeptides (e.g., colistin), and derivatives thereof. In a particular embodiment, the antibiotic is vancomycin or metronidazole. In a particular embodiment of the invention, a narrow spectrum macrocyclic antibiotic drug is used (e.g. fidaxomicin).

The non-toxigenic Clostridium may be administered to a host (e.g., human or animal) in a composition with a pharmaceutically acceptable carrier. For example, the non-toxigenic Clostridium (e.g., spores thereof) may be formulated with a pharmaceutically acceptable carrier or suitable mixtures thereof. The concentration of the non-toxigenic Clostridium in the chosen medium may be varied and the medium may be chosen based on the desired route of administration of the pharmaceutical preparation. Except insofar as any conventional media or agent is incompatible with the non-toxigenic Clostridium, its use in the pharmaceutical preparation is contemplated.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Appropriate concentrations for alleviation of a particular pathological condition may be determined by dosage concentration curve calculations, as known in the art. The dose and dosage regimen of the non-toxigenic Clostridium that are suitable for administration to a particular patient may be determined by a physician considering the patient's age, sex, weight, general medical condition, and the specific condition for which the non-toxigenic Clostridium is being administered and the severity thereof. For example, dosage units may be proportionately increased or decreased based on the weight of the patient. The physician may also take into account the route of administration, the pharmaceutical carrier, and the biological activity of the administered non-toxigenic Clostridium. An embodiment of the invention includes a route of administration via rectal enema.

Pharmaceutical compositions containing a non-toxigenic Clostridium as the active ingredient in intimate admixture with a pharmaceutically acceptable carrier can be prepared according to conventional pharmaceutical compounding techniques. The carrier may take a wide variety of forms depending on the form of preparation desired for administration. The non-toxigenic Clostridium may be administered as cells or spores. When spores are utilized, they may be lyophilized. The compositions of the present invention can be prepared, for example, in liquid form, or can be in dried powder form. Dosage forms for oral administration include, without limitation, tablets (e.g., coated and uncoated, chewable), gelatin capsules (e.g., soft or hard), pills, time-release capsules, lozenges, troches, solutions, emulsions, suspensions, syrups, elixirs, powders/granules (e.g., reconstitutable or dispersible) gums, and effervescent tablets. Corresponding dosage forms for a suppository or enema formulation are also encompassed herein. In a particular embodiment, the composition is formulated as an oral suspension, such as an oral aqueous suspension comprising polysorbate 80.

### Definitions

The term “treat” as used herein refers to any type of treatment that imparts a benefit to a patient afflicted with a disease, including improvement in the condition of the patient (e.g., in one or more symptoms), delay in the progression of the condition, etc. In a particular embodiment, the treatment of a Clostridium associated disease results in at least an inhibition/reduction in diarrhea.

The phrase “effective amount” refers to that amount of therapeutic agent that results in an improvement in the patient's condition.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., thimersol, benzyl alcohol), anti-oxidant (e.g., ascorbic acid, sodium metabisulfite), solubilizer (e.g., Tween™ 80, polysorbate 80), emulsifier, buffer (e.g., tris HCl, acetate, phosphate), water, aqueous solutions, oils, bulking substance (e.g., lactose, mannitol), excipient, auxilliary agent or vehicle with which an active agent of the present invention is administered. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, 20th Edition, (Lippincott, Williams and Wilkins), 2000; Liberman, et al., Eds., Pharmaceutical Dosage Forms, Marcel Decker, New York, N.Y., 1980; and Kibbe, et al., Eds., Handbook of Pharmaceutical Excipients (3rd Ed.), American Pharmaceutical Association, Washington, 1999.

The term “isolated” may refer to protein, nucleic acid, compound, or cell that has been sufficiently separated from the environment with which it would naturally be associated, so as to exist in “substantially pure” form. “Isolated” does not necessarily mean the exclusion of artificial or synthetic mixtures with other compounds or materials, or the presence of impurities that do not interfere with the fundamental activity, and that may be present, for example, due to incomplete purification.

The term “non-toxigenic”, as used herein refers, to a strain of Clostridium bacteria that are substantially deficient for toxin production (e.g., produce less than about 5%, 3%, 1%, 0.5% or less toxins compared to toxigenic Clostridium) or fail to produce any toxin (e.g., strains that lack one or more genes for toxin production). The term “non-toxigenic C. difficile” denotes C. difficile that are substantially deficient for Toxin A, Toxin B, and Binary Toxin production or fail to produce any Toxin A, Toxin B and Binary Toxin (e.g., strains that lack one or more genes for Toxin A, Toxin B, and Binary Toxin production).

The following example is provided to illustrate certain embodiments of the invention. It is not intended to limit the invention in any way.

### EXAMPLE

A phase 1 study was conducted to assess the safety and tolerability of an oral suspension of spores of a non-toxigenic strain of C. difficile (VP20621) in healthy adult subjects. VP20621 (10⁴, 10⁶, or 10⁸ spores) or placebo were administered as a single dose to subjects age 18-45 (Group 1) or ≧60 years of age (Group 2). In Group 3, an oral suspension of 10⁸ spores or placebo was administered twice daily for five days to patients≧60 years in age. In Group 4, subjects≧60 years of age received 5 days of oral vancomycin followed by 14 days of once daily VP20621 (10⁴, 10⁶, or 10⁸ spores) or placebo. All subjects were followed through day 28. C. difficile stool cultures were performed at various time points. C. difficile isolates were tested for the production of toxin by enzyme immunoassay.

VP20621 was well tolerated. No serious or severe adverse events (AEs) were reported and no subjects discontinued drug study. In Groups 1-3, there were no subjects with AEs of diarrhea or change in stool form or frequency. In Group 4 during pre-treatment with vancomycin, 16% had a gastrointestinal adverse effect and 7% of subjects had mild diarrhea. During subsequent dosing with study drug, gastrointestinal AEs were reported in 22% (6/27) VP20621 subjects (all doses) and 33% (3/9) placebo subjects. 3 (11%) VP20621 subjects reported mild loose or watery stool on a single study day that did not require treatment and resolved despite continued dosing. Groups 1 and 2: no C. difficile was cultured from stool samples. Group 3: non-toxigenic C. difficile was detected in stool cultures on various days from all active subjects between days 2 and 7. Group 4: non-toxigenic C. difficile was detected in stool cultures from all active subjects during the dosing period and in some subjects on days 14 and/or 28. Surprisingly, non-toxigenic C. difficile was also detected in stool cultures from placebo subjects in the 10⁸ cohorts.

This phase 1 study showed VP20621 to be well tolerated at all doses tested in younger and older volunteer subjects. Pretreatment with oral vancomycin created a susceptible environment for colonization, mimicking the clinical situation in which most C. difficile infections occur. Infection with non-toxigenic C. difficile was detected in placebo subjects who were not administered the non-toxigenic C. difficile directly.

### Introduction

Although current therapies for the treatment of Clostridium difficile infection (CDI) are effective in the majority of patients, 20-30% of patients experience a recurrence of CDI. New therapies are needed for the treatment of recurrence of CDI, and ultimately for the prevention of CDI.

Numerous studies utilizing the hamster model of CDI have demonstrated that colonization of hamsters with non-toxigenic C. difficile could safely prevent either recurrence of CDI or primary CDI (Sambol et al. (2002) J. Infect. Dis., 186:1781-9; 6. Merrigan et al. (2003) J. Infect. Dis., 188:1922-7; Merrigan et al., Abstract #K-1092, The 42^{nd} ICAAC, San Diego, Calif., Sep. 27-30, 2002; Merrigan et al. (2009) Inter. J. Antimicrob. Agents, 33:S46-S50; Nagaro et al., “Non-toxigenic Clostridium difficile (CD) protects hamsters against historic and epidemic toxigenic “BI” strains.” Fifth International Meeting on the Molecular Biology and Pathogenesis of the Clostridia (ClostPath 2006). Jun. 21-25, 2006, Nottingham, UK). In addition, a review of four epidemiological studies of asymptomatic C. difficile colonization in hospitalized patients (Shim et al. (1998) Lancet, 351:633-636) provided evidence for not only the safety of asymptomatic C. difficile colonization in humans with a non-toxigenic strain, but also the potential of asymptomatic colonization to prevent CDI.

VP 20621 is a formulation of spores of a non-toxigenic strain of C. difficile. Genetic analyses confirmed that this strain lacks the genes for Toxin A, Toxin B, and Binary Toxin. In addition, preclinical safety testing confirmed that this strain demonstrated a negative finding in an enzyme immunoassay for Toxin A, a negative finding in the cell cytotoxicity assay for Toxin B, and produced no enterotoxicity in the rabbit ileal loop assay.

In Phase 1 evaluations, it was critical to evaluate the safety and tolerability of VP 20621 administered to older subjects (≧60 years of age) because older individuals represent the highest risk group for colonization with toxigenic strains of C. difficile and subsequent development of CDI. Initial results demonstrated that single, escalating doses of VP 20621 (10⁴, 10⁶, 10⁸ spores) and multiple doses (10⁸ spores BID for 5 days) were safe in healthy subjects≧60 years (Tatarowicz et al., “Safety and tolerability of an oral suspension of VP 20621, spores of a non-toxigenic C. difficile strain; first in human administration to healthy adult subjects.” Tenth Biennial Congress of the Anaerobe Society of the Americas. Jul. 7-10, 2010. Philadelphia, Pa.). This portion of a Phase 1 study evaluated the safety and efficacy of escalating doses of spores (10⁴, 10⁶, 10⁸ spores) administered daily for 14 days in healthy subjects≧60 years of age who were pretreated with oral vancomycin for 5 days.

### Materials and Methods

This study was conducted at a single investigative site in Switzerland. At the screening visit, subjects were issued a stool diary and were instructed to record and track information regarding bowel habits from Day −12 through admission to the study unit on Day −6.

### Inclusion/Exclusion Criteria

Inclusion: Subjects≧60 years of age needed to be healthy, could not have taken any prescription or non-prescription drugs during the study period, and recorded at least 4 bowel movements in the stool diary (Day −12 through admission to the study unit on Day −6).
Exclusion: Subjects were excluded if they had a known gastrointestinal disease or disorder affecting the regular function of the lower gastrointestinal tract, taken any antibiotics from 3 months prior to screening visit through randomization, or had recorded 4 or more bowel movements on any one day in the stool diary (Day −12 through admission to the study unit on Day −6).

### In-Clinic Period (Day −6 Through Day 14)

All subjects were admitted to the study unit on Day −6. Study personnel recorded and tracked each subject's bowel habits during the in-clinic period. Daily stool samples were collected from Day −6 (baseline) through Day 14. All subjects were discharged from the study unit on Day 14.

### Study Drug Dosing

Days −5 to −1: Oral vancomycin 125 mg QID
Days 1-14: VP 20621 (10⁴, 10⁶, 10⁸ spores) or matching placebo once daily. Purified VP 20621 (spores of a non-toxigenic strain of C. difficile) were produced in a liquid culture medium free of animal-derived components. VP 20621 was administered as an oral liquid suspension. The potency of the drug is based on the viable count of the spores.

### Follow-Up Period and End-of-Study (Days 21 and 28)

All subjects returned to the clinical for follow-up on Day 21 and for the end-of study visit on Day 28. Stool samples were collected during both visits.

### C. Difficile Stool Cultures

Stool cultures were performed at Viollier AG (Basel, Switzerland). Stool cultures were inoculated onto cycloserine-cefoxitin-fructose agar plates (CLO agar; bioMerieux; Marcy l'Etoile, France) and incubated for 48 hours under anaerobic conditions. C. difficile was identified by fluorescence (366 nm) colonial morphology (yellowish colonies with frayed edges), cresol-like odor, and MALDI-TOF profile. Select isolates were tested for the presence of C. difficile Toxins A and B in culture supernatants. Toxins were detected using the C. difficile Tox AB II™ kit (Techlab; Blacksburg, Va.).

In addition, blinded stool samples from Days 21 and 28 were sent to the laboratory of Dr. Dale Gerding (Hines VA Hospital, Hines, Ill.) for culture. Samples were either inoculated on taurocholate-cycloserine-cefoxitin-fructose agar (TCCFA) agar or treated with ethanol prior to inoculation on TCCFA agar. Colonies resembling C. difficile were selected for restriction endonuclease analysis (REA) genotyping.

For data analysis, isolation of C. difficile from either laboratory was considered a positive culture.

### C. Difficile Genotyping

Selected C. difficile isolates were genotyped using a pulsed-field gel electrophoresis (PFGE) assay or by REA. Isolates with banding patterns consistent with the VP 20621 control were considered to be VP 20621.

In general, only the first and last C. difficile isolates from a subject were genotyped. It was assumed that if the genotyping of those isolates matched, all isolates obtained at interim timepoints would also be of the same genotype.

### Results

### Adverse Events Reported During Vancomycin Pre-Treatment Period:

Forty-three (43) subjects were pre treated with vancomycin to ensure that a sufficient number of subjects would be available for randomization to achieve the planned target sample size of 36. Twelve (28%) of the 43 subjects had adverse events during the vancomycin pre treatment period (Table 1).

TABLE 1
Adverse Events Reported During Vancomycin Pre-Treatment (N = 43).

| | |
|---|---|
| Gastrointestinal AEs | Other AEs |

| | | | |
|---|---|---|---|
| Diarrhea | 3 (7%) | Dizziness | 2 (5%) |
| Abdominal pain | 2 (5%) | Dry skin | 1 (2%) |
| Abdominal distention | 1 (2%) | Malaise | 1 (2%) |
| Flatulence | 1 (2%) | Pruritis | 1 (2%) |
| Gingival bleeding | 1 (2%) | Rash | 1 (2%) |
| Oral paresthesia | 1 (2%) | Dry skin | 1 (2%) |
| Toothache | 1 (2%) | | |
| Vomiting | 1 (2%) | | |

### Demographics:

Thirty-six subjects were randomized to receive VP 20621 or placebo. Demographics of randomized subjects are shown in Table 2.

TABLE 2
Demographics of Dosing Cohorts.

| | | | VP 20621 Cohorts | | | | | | | |
|---|---|---|---|---|---|---|---|---|---|---|
| | Placebo | | 10⁴ | | 10⁶ | | 10⁸ | | All Doses | |
| All treated | 9 | | 9 | | 9 | | 9 | | 27 | |
| subjects, N | | | | | | | | | | |
| Age (years) | | | | | | | | | | |
| Mean | 64 | (3.7) | 66 | (3.8) | 63 | (2.7) | 64 | (4.2) | 64 | (3.7) |
| (SD) | | | | | | | | | | |
| Median | 64 | (61, 73) | 66 | (60, 73) | 62 | (60, 69) | 62 | (60, 73) | 64 | (60, 73) |
| (Min, Max) | | | | | | | | | | |
| Gender, N | | | | | | | | | | |
| (%) | | | | | | | | | | |
| Female | 5 | (55.6) | 1 | (11.1) | 3 | (33.3) | 4 | (44.4) | 8 | (29.6) |
| Male | 4 | (44.4) | 8 | (88.9) | 6 | (66.7) | 5 | (55.6) | 19 | (70.4) |
| Race, N (%) | | | | | | | | | | |
| White | 9 | (100) | 9 | (100) | 9 | (100) | 9 | (100) | 27 | (100) |
| Body | | | | | | | | | | |
| Weight, kg | | | | | | | | | | |
| Female | | | | | | | | | | |
| Mean | 70 | (9.3) | 71 | (—) | 71 | (4.2) | 68 | (8.3) | 70 | (6.0) |
| (SD) | | | | | | | | | | |
| Male | | | | | | | | | | |
| Mean | 82 | (4.0) | 83 | (10.4) | 85 | (10.7) | 82 | (1.7) | 84 | (8.7) |
| (SD) | | | | | | | | | | |

### Treatment-Emergent Adverse Events:

Following multiple escalating doses of study drug (placebo or VP 20621 QD for 14 days), treatment-emergent adverse events were reported by 5/9 (56%) subjects who received placebo and 12/27 (44%) subjects who received any dose of VP 20621. Gastrointestinal adverse events are summarized in Table 3.

TABLE 3
Treatment-emergent Gastrointestinal Adverse Events.

| | Group 4 | | | | |
|---|---|---|---|---|---|
| | | VP 20621 Cohorts | | | |
| Adverse Events | Placebo | 10⁴ | 10⁶ | 10⁸ | All Doses |
| Treated subjects, N | 9 | 9 | 9 | 9 | 27 |
| N (%) with ≧1 | 3 (33%) | 4 (44%) | 2 (22%) | 0 | 6 (22%) |
| Gastrointestinal TEAE | | | | | |
| Diarrhea* | 0 | 2 (22%) | 1 (11%) | 0 | 3 (11%) |
| Dyspepsia | 1 (11%) | 1 (11%) | 1 (11%) | 0 | 2 (7%) |
| Abdominal discomfort | 0 | 0 | 1 (11%) | 0 | 1 (4%) |
| Abdominal pain upper | 0 | 0 | 1 (11%) | 0 | 1 (4%) |
| Constipation | 0 | 1 (11%) | 0 | 0 | 1 (4%) |
| Flatulence | 2 (22%) | 0 | 0 | 0 | 0 |
| Gingival pain | 1 (11%) | 0 | 0 | 0 | 0 |

TEAE = treatment-emergent adverse event.
*Mild episodes of watery or loose stool on Day 6 or 8; no treatment required; resolved despite continued dosing.

A summary of adverse events considered related to study drug is provided in Table 4.

TABLE 4
Treatment-emergent Adverse Events Related to Study Drug.

| | Group 4 | | | | |
|---|---|---|---|---|---|
| | | VP 20621 Cohorts | | | |
| Adverse Events | Placebo | 10⁴ | 10⁶ | 10⁸ | All Doses |
| Treated subjects, N | 9 | 9 | 9 | 9 | 27 |
| N (%) with ≧1 TEAE | 2 (22%) | 2 (22%) | 3 (33%) | 0 | 5 (19%) |
| related to study drug | | | | | |
| Diarrhea* | 0 | 2 (22%) | 1 (11%) | 0 | 3 (11%) |
| Abdominal discomfort | 0 | 0 | 1 (11%) | 0 | 1 (4%) |
| Abdominal pain upper | 0 | 0 | 1 (11%) | 0 | 1 (4%) |
| Chest discomfort | 0 | 0 | 1 (11%) | 0 | 1 (4%) |
| Dyspepsia | 1 (11%) | 0 | 1 (11%) | 0 | 1 (4%) |
| Pruritus | 0 | 0 | 1 (11%) | 0 | 1 (4%) |
| Rash | 0 | 0 | 1 (11%) | 0 | 1 (4%) |
| Flatulence | 2 (22%) | 0 | 0 | 0 | 0 |

TEAE = treatment-emergent adverse event.
*Mild episodes of watery or loose stool on Day 6 or 8; no treatment required; resolved despite continued dosing.

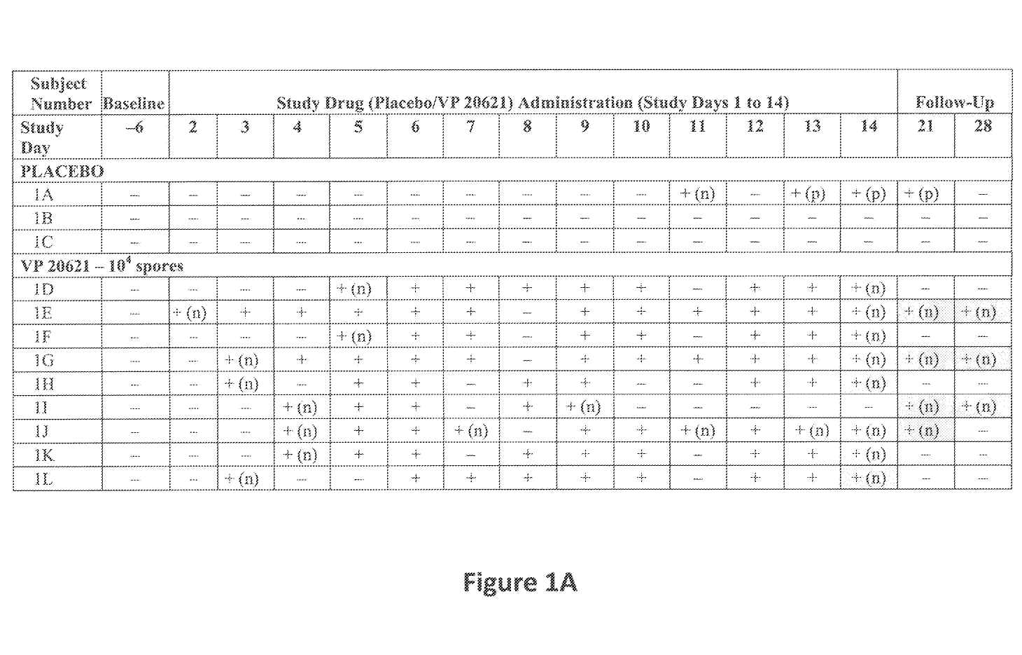

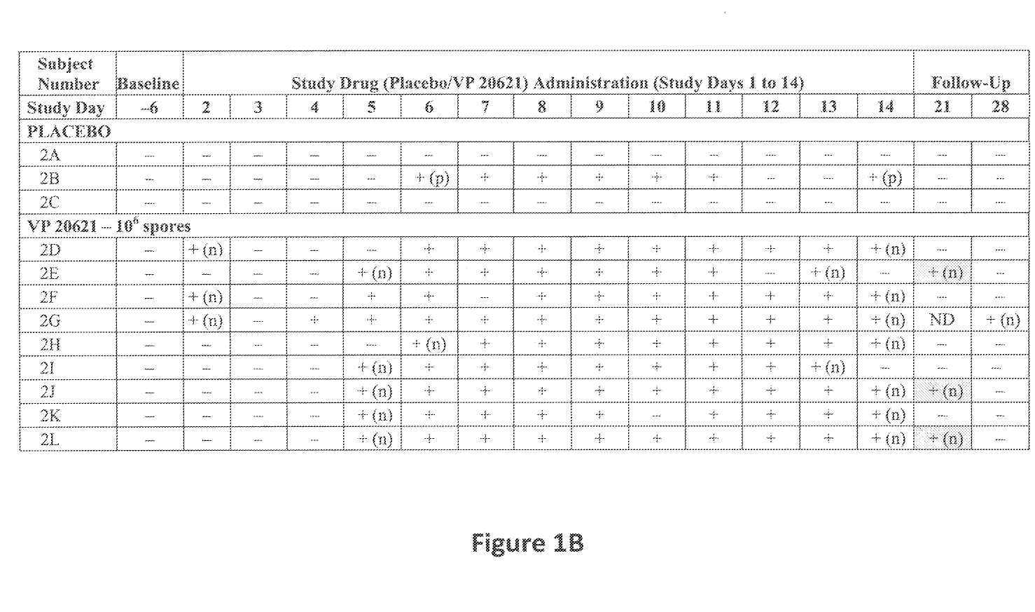

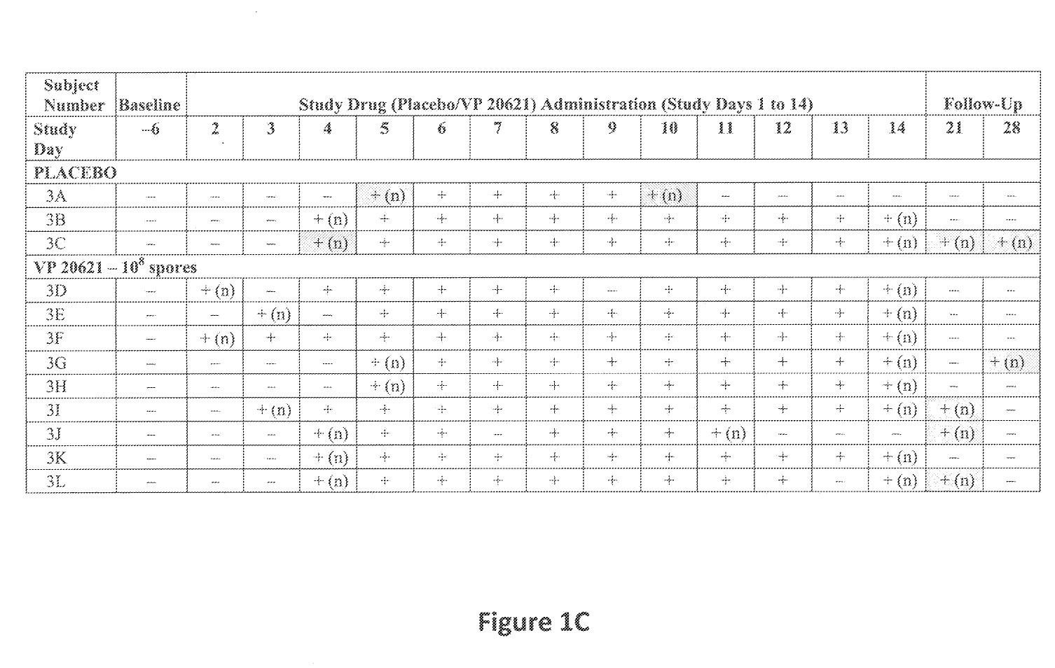

FIGS. 1A-1C provide the C. difficile stool culture results for cohort 1 (placebo or 10⁴ spores), cohort 2 (placebo or 10⁶ spores), and cohort 3 (placebo or 10⁸ spores), respectively.

### Discussion

VP 20621 was well tolerated. There were no serious or severe adverse events, and no subjects were discontinued from study drug due to an adverse event. Overall there was no evidence that the type or severity of events were dose-dependent.

During pre-treatment with vancomycin, 3/43 (7%) subjects had mild diarrhea or loose/watery stools. During subsequent dosing with study drug, 3/27 (11%) VP 20621 subjects reported mild loose or watery stools on a single study day that did not require treatment and resolved despite continued dosing. These subjects did not have any unique pattern in their stool culture results. The only other GI adverse event reported in more than one VP 20621 subject was mild dyspepsia (2/27; 7%).

VP 20621 was isolated from stool cultures during the dosing period from all subjects who received oral vancomycin and VP 20621. In addition, VP 20621 was isolated from stool cultures at least 1 week after the last dose of spores in 12 of the 27 subjects who received spores, indicating that these subjects were colonized with VP 20621. Previous evaluation of VP 20621 in healthy subjects without prior treatment with oral vancomycin found that no subjects became colonized with VP 20621 (Tatarowicz et al. “Safety and tolerability of an oral suspension of VP 20621, spores of a non-toxigenic C. difficile strain; first in human administration to healthy adult subjects.” Tenth Biennial Congress of the Anaerobe Society of the Americas. Jul. 7-10, 2010, Philadelphia, Pa.). These data indicate that disruption of the gut microbiota with oral vancomycin created an environment suitable for colonization with VP 20621.

Toxin-positive C. difficile was isolated from two subjects who received placebo after initial treatment with oral vancomycin. Similar observations were observed in studies when antibiotics were given to healthy subjects (Ambrose et al. (1985) J. Antimicrob. Chemother., 15:319-26; Finegold et al. (1987) Antimicrob. Agents Chemother., 31:443-6; Brismar et al. (1993) Infection, 21:373-5; Chachaty et al. (1993) Antimicrob. Agents Chemother., 37:1432-5). These subjects had no GI adverse events.

VP 20621 was isolated from two placebo subjects in the cohort receiving 10⁸ spores (Cohort 3) with positive stool cultures on numerous days during the dosing period, similar to the results for subjects who received VP 20621. These results are due to exposure to VP 20621 spores within the study site through contact with the other study subjects in that cohort or through contact with items within the shared living facilities. These subjects had no adverse GI adverse events except for 1 subject with abdominal distension that had started during pretreatment with vancomycin prior to starting VP 20621.

In this Phase 1 trial, multiple doses of VP 20621 administered after oral vancomycin were well tolerated at all dose levels administered; there were no serious or severe adverse events, and no subjects were discontinued from study drug due to an adverse event. VP 20621 was detected in stool cultures at one or more timepoints in all subjects who received VP 20621. These data indicate that the VP 20621 strain of C. difficile can colonize the GI tract of patients with disrupted GI microbiota who are at risk for acquiring toxigenic C. difficile, thereby preventing CDI.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method of inhibiting disease caused by Clostridium in a subject, said method comprising:
a) providing a first subject that has been administered a non-toxigenic strain of said Clostridium, and that retains an amount of said non-toxigenic Clostridium effective to cause shedding of said non-toxigenic Clostridium by said first subject;
b) administering at least one antibiotic to a second subject; and
c) exposing said second subject to said first subject, wherein the exposure of said second subject to said first subject results in the colonization of the gastrointestinal tract of said second subject by said non-toxigenic Clostridium.

2. The method of claim 1, wherein said non-toxigenic Clostridium is C. difficile or C. butyricum.

3. The method of claim 1, wherein said non-toxigenic strain of Clostridium is a C. difficile strain selected from the group consisting of M, M3, M23, T, T7, C, P, S, and AP.

4. The method of claim 3, wherein said non-toxigenic C. difficile strain is M3.

5. The method of claim 1, wherein said non-toxigenic strain of Clostridium is C. butyricum MIYAIRI 588.

6. The method of claim 1, wherein said disease is cause by C. difficile.

7. The method of claim 1, wherein said second subject is human.

8. The method of claim 7, wherein said first and second subjects are humans.

9. The method of claim 1, wherein said antibiotic is vancomycin or fidaxomicin.

10. The method of claim 1, wherein said exposure occurs within about 48 hours of step b).

11. The method of claim 10, wherein said exposure occurs within about 24 hours of step b).

12. The method of claim 1, wherein said colonization by said non-toxigenic Clostridium occurs within about 72 hours of step c).

13. The method of claim 1, wherein said exposure occurs without direct physical contact between said subjects.

14. The method of claim 1, wherein said exposure occurs for a period from about 1 hour to about 2 days.

15. A method of inhibiting disease caused by Clostridium in a subject, said method comprising:
a) administering at least one antibiotic to said subject; and
b) contacting the environment of said subject with an effective amount of a non-toxigenic strain of said Clostridium, wherein said subject is maintained in said environment for a time sufficient to allow the colonization of the gastrointestinal tract of said subject by said non-toxigenic Clostridium.

16. The method of claim 15, wherein said Clostridium is C. difficile or C. butyricum.

17. The method of claim 15, wherein said non-toxigenic strain of Clostridium is a C. difficile strain selected from the group consisting of M, M3, M23, T, T7, C, P, S, and AP.

18. The method of claim 17, wherein said C. difficile strain is M3.

19. The method of claim 15, wherein said non-toxigenic strain of Clostridium is C. butyricum MIYAIRI 588.

20. The method of claim 15, wherein said antibiotic is vancomycin or fidaxomicin.

21. The method of claim 15, wherein step b) occurs within about 48 hours of step a).

22. The method of claim 21, wherein step b) occurs within about 24 hours of step a).

23. The method of claim 15, wherein said colonization by said non-toxigenic Clostridium occurs within about 72 hours of step b).

24. The method of claim 15, wherein said subject is maintained in said environment for about 1 hour to about 2 days.

25. The method of claim 15, wherein said environment is contacted with a composition comprising a non-toxigenic strain of said Clostridium and at least one pharmaceutically acceptable carrier.

26. A composition comprising a pharmaceutically unacceptable carrier and at least one non-toxigenic strain of Clostridium.

27. The composition of claim 26, wherein said non-toxigenic strain of Clostridium is a C. difficile strain selected from the group consisting of M, M3, M23, T, T7, C, P, S, and AP.

28. A method of producing a non-toxigenic strain of C. difficile comprising administering to an animal host a sufficient quantity of said non-toxigenic strain of C. difficile spores to induce colonization of the gastrointestinal tract of said host by said spores, thereby causing shedding of said spores by said host.

29. The method of claim 28, wherein said non-toxigenic strain of C. difficile is a strain selected from the group consisting of M, M3, M23, T, T7, C, P, S, and AP.

30. The method of claim 29, wherein said non-toxigenic C. difficile strain is M3.

31. The method of claim 28, further comprising exposing said host to a patient in need of treatment for a disease caused by Clostridium, thereby effecting transfer of said non-toxigenic C. difficile spores from said host to said patient.

32. The method of claim 28, further comprising exposing said host to a treatment site in which a patient in need of treatment for a disease caused by Clostridium receives said treatment, thereby effecting transfer of said non-toxigenic C. difficile spores from said host to said treatment site.

33. The method of claim 28, wherein said host is treated with an antibiotic prior to administration of said non-toxigenic C. difficile spores.

34. The method of claim 33, wherein said antibiotic is vancomycin or fidaxomicin.

35. The method of claim 28, wherein said host is a human host.

36. A method of protecting a patient undergoing medical treatment at a treatment site from acquiring a disease caused by a Clostridial infection while present at said site, the method comprising dispersing a non-toxic strain of C. difficile at said site in an amount sufficient to be transferred to said patient, thereby effecting colonization of the gastrointestinal tract of said patient by said non-toxigenic strain of C. difficile.

37. The method of claim 36, wherein said non-toxigenic strain of C. difficile is a strain selected from the group consisting of M, M3, M23, T, T7, C, P, S, and AP.

38. The method of claim 37, wherein said C. difficile strain is M3.

39. A method of reducing the risk that a medical treatment site will induce a disease caused by a Clostridial infection in a patient upon undergoing treatment at said site, the method comprising dispersing a non-toxic strain of C. difficile at said site in an amount that is sufficient to be transferred to a patient exposed to said site, to thereby effect colonization of the gastrointestinal tract of said patient by said non-toxigenic strain of C. difficile.

40. The method of claim 38, wherein said non-toxigenic strain of C. difficile is a strain selected from the group consisting of M, M3, M23, T, T7, C, P, S, and AP.

41. The method of claim 39, wherein said C. difficile strain is M3.
